(19) United States
(12) Patent Application Publication
(10) Pub. No.: US 2026/0315361 A1
(43) Pub. Date: Oct. 15, 2026

(54) STABILIZED FRAGRANCE COMPOSITIONS AND USES THEREOF

(71) Applicant: FIRMENICH SA, Satigny (CH)

(72) Inventors: Tieu-Vey LAM, Athis Mons (FR); Wolfgang FIEBER, Ornex (FR); Anais KOLB, Paris (FR); Barbara BUCHS, Neuville sur Oise (FR); Sylvie BRETON, Neuville sur Oise (FR)

(73) Assignee: FIRMENICH SA, Satigny (CH)

(21) Appl. No.: 19/116,615

(22) PCT Filed: Sep. 22, 2023
(86) PCT No.: PCT/EP2023/076226

§ 371 Date: Mar. 28, 2025

(30) Foreign Application Priority Data
Oct. 7, 2022 (EP) 22200191.9

(51) Int. Cl. A61K 8/67; A61K 8/34; A61K 8/365; A61K 8/46; A61K 8/55; A61K 8/86; A61Q 13/00

(52) U.S. Cl. CPC A61K 8/678; A61K 8/347; A61K 8/365; A61K 8/46; A61K 8/55; A61K 8/86; A61Q 13/00; A61K 2800/522

(57) ABSTRACT

The present disclosure relates to fragrance compositions comprising a stabilizing mixture. The present disclosure also relates to consumer products containing the stabilized fragrance composition.

### CROSS REFERENCE TO RELATED APPLICATIONS

This application claims the benefit of priority of European Application No. 22200191.9, filed Oct. 7, 2022, which is hereby incorporated by reference as though set forth herein in its entirety.

### FIELD OF THE DISCLOSURE

The present disclosure relates to fragrance compositions comprising a stabilizing mixture. The present disclosure also relates to consumer products containing the stabilized fragrance composition.

### BACKGROUND OF THE DISCLOSURE

Many perfume ingredients, fragrance oils, formulated perfumes, fine fragrance products, hydroalcoholic products, body care products, cosmetic products, perfumery raw materials (such as essential oils, natural extracts, and synthetic ingredients) can undergo oxidation when exposed to the atmosphere, heat and/or light. Oxidation can lead to changes in organoleptic properties and/or the appearance of the perfume ingredient, formulated perfume, formulated body care product, essential oil, or natural extract.

Such oxidation of perfume ingredients, formulated perfumes, body care products, cosmetic products, and/or perfumery raw materials often leads to olfactive deviation, discoloration, turbidity or cloudiness from precipitation or sedimentation, pH change, chemical degradation, formation of skin irritants, among others. In ethanol-containing formulations, such as eau de toilettes, oxidation of ethanol results in the formation of acetaldehyde, which reacts with ethanol to form the corresponding acetal, or diethoxyethane, which exhibits a characteristic, but undesirable, odor.

One way to address the problem of oxidative stability is the addition of one or more stabilizers to fragrance concentrates. However, this strategy is not without its own challenges. One major challenge is the compatibility of the stabilizing ingredients with the perfumery ingredients or raw materials or even compatibility between the stabilizing ingredients arising from different polarities and lack of common solvents. Another challenge arises from recent and proposed changes in legislation limiting the use of certain ingredients or requiring the use of more environmentally friendly ingredients in the perfumery industry. Yet another challenge is the need to change with market forces as the industry moves towards ingredients originating from natural sources, such as renewable carbon sources.

Still another challenge is the compatibility of the perfumery ingredients, raw materials, or fragrance oils containing the stabilizing ingredients when incorporated into the final product application. Ideally, the stabilizing ingredients should protect the fragrance oil from its production to its use and subsequently protect the fragrance in the final product with sufficient efficiency.

Therefore, there is an ongoing need for fragrance compositions capable of mitigating olfactive deviation, discoloration, turbidity or cloudiness from precipitation or sedimentation, pH change, chemical degradation, and/or formation of skin irritants, while also addressing one or more of the aforementioned challenges.

### SUMMARY OF THE DISCLOSURE

The following aspects of the present disclosure seek to address one or more of the problems described hereinabove.

In a first aspect, the present disclosure relates to a fragrance composition comprising:
a) a fragrance oil, and
b) a stabilizing mixture comprising at least 2 ingredients, typically at least 3 ingredients, each having a function selected from the group consisting of antioxidant, metal chelator, pH regulator, and POV remediant,
wherein the at least 2 ingredients, typically at least 3 ingredients, do not have the same function, and
wherein the composition contains less than about 1 wt % water, relative to the total weight of the composition.

In a second aspect, the present disclosure relates to a solution comprising:
one or more solvents, and
at least 2 ingredients, typically at least 3 ingredients, each having a function selected from the group consisting of antioxidant, metal chelator, pH regulator, and POV remediant,
wherein the at least 2 ingredients, typically at least 3 ingredients, do not have the same function.

In a third aspect, the present disclosure relates to a water-in-oil microemulsion comprising:
a) a discontinuous aqueous phase comprising water and one or more water-soluble compounds each having a function selected from the group consisting of antioxidant, metal chelator, pH regulator, POV remediant, and combinations thereof;
b) a continuous oil phase comprising one or more oil-soluble compounds each having a function selected from the group consisting of antioxidant, metal chelator, pH regulator, POV remediant, and combinations thereof;
c) a surfactant; and
d) a water-miscible solvent.

In a fourth aspect, the present disclosure relates to a consumer product comprising the fragrance composition, the solution, or the water-in-oil microemulsion described herein.

In a fifth aspect, the present disclosure relates to use of the stabilizing mixture as defined herein or the solution or the water-in-oil microemulsion described herein to enhance the oxidative stability of a fragrance oil in a fragrance composition or a consumer product.

### DETAILED DESCRIPTION

As used herein, the terms “a”, “an”, or “the” means “one or more” or “at least one” unless otherwise stated.

While compositions and methods are described in terms of “comprising,” “containing,” or “including” various components or steps, the compositions and methods can also “consist essentially of” or “consist of” the various components, substances and steps. As used herein the term “consisting essentially of” shall be construed to mean including the listed components, substances or steps and such additional components, substances or steps which do not materially affect the basic and novel properties of the composition or method. In some embodiments, a composition in accordance with embodiments of the present disclosure that “consists essentially of” the recited components or substances does not include any additional components or substances that alter the basic and novel properties of the composition.

Unless defined otherwise, all technical and scientific terms used herein have the same meaning as is commonly understood by one of skill in the art to which this specification pertains.

It should be understood that any numerical range recited herein is intended to include all sub-ranges subsumed therein. For example, a range of “1 to 10” is intended to include all sub-ranges between and including the recited minimum value of 1 and the recited maximum value of 10; that is, having a minimum value equal to or greater than 1 and a maximum value of equal to or less than 10. Because the disclosed numerical ranges are continuous, they include every value between the minimum and maximum values. Unless expressly indicated otherwise, the various numerical ranges specified in this application are approximations.

As used herein, and unless otherwise indicated, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

Throughout the present disclosure, various publications may be incorporated by reference. Should the meaning of any language in such publications incorporated by reference conflict with the meaning of the language of the present disclosure, the meaning of the language of the present disclosure shall take precedence, unless otherwise indicated.

Throughout the present disclosure, various chemical names and structures may be recited. Unless otherwise stated, any stereoisomers, such as enantiomers, diastereomers, anomers, epimers, and the like; and geometric isomers, such as cis/trans or E/Z isomers, of the recited chemical name or structure are contemplated. As would be understood by those of ordinary skill in the art, stereoisomers may possess one stereocenter, giving rise to enantiomers, or more than one stereocenter, giving rise to diastereomers, each stereocenter having one of two different stereochemistries (i.e., R or S). Enantiomers may be characterized by their ability to rotate oncoming plane-polarized light to the right, designated as dextrorotatory, “(+)” or “D”, or to the left, designated as levorotatory, “(−)” or “L”. Enantiomers may exist as racemic mixtures or scalemic mixtures. Geometric isomers refer to isomers in which the spatial relationship of atoms around a double bond are different, typically designated E or Z according to conventional understanding in the chemical art. Geometric isomers may also exist as mixtures of E and Z isomers. All of the aforementioned isomeric variations of the chemical names or structures recited herein are included.

In the first aspect, the present disclosure relates to a fragrance composition comprising:
a) a fragrance oil, and
b) a stabilizing mixture comprising at least 2 ingredients, typically at least 3 ingredients, each having a function selected from the group consisting of antioxidant, metal chelator, pH regulator, and POV remediant,
wherein the at least 2 ingredients, typically at least 3 ingredients, do not have the same function, and
wherein the composition contains less than about 1 wt % water, relative to the total weight of the composition.

As used herein, an “antioxidant” and an “ingredient having an antioxidant function” are used interchangeably. Similarly, a “metal chelator” and an “ingredient having a metal chelator function” are used interchangeably. A “pH regulator” and an “ingredient having pH regulator function” are used interchangeably. A “POV remediant” and an “ingredient having a POV remediant function” are used interchangeably.

In an embodiment, the stabilizing mixture comprises at least 3 ingredients, each having a function selected from the group consisting of antioxidant, metal chelator, pH regulator, and POV remediant.

In another embodiment, the stabilizing mixture comprises at least one antioxidant and at least one metal chelator.

In yet another embodiment, the stabilizing mixture comprises two antioxidants and two metal chelators.

In an embodiment, the stabilizing mixture comprises at least one antioxidant, at least one metal chelator, and at least one pH regulator.

In another embodiment, the stabilizing mixture comprises one antioxidant, two metal chelators, and two pH regulators.

In yet another embodiment, the stabilizing mixture comprises at least one antioxidant, at least one pH regulator, and at least one POV remediant.

In still another embodiment, the stabilizing mixture comprises at least one antioxidant, at least one metal chelator, at least one pH regulator, and at least one POV remediant.

As used herein, the antioxidant is any compound or combination of compounds capable of scavenging free radicals formed during oxidation. Exemplary antioxidants include, but are not limited to, ascorbic acid, gallic acid, ferulic acid, caffeic acid, tannic acid, chlorogenic acid, ellagic acid, rosmarinic acid, a polyphenol, tetramethylhydroxypiperidinol, vitamin E, α-tocopherol, β-tocopherol, γ-tocopherol, δ-tocopherol, ascorbyl palmitate, ascorbyl glucoside, natural extracts, typically rosemary extract, curcumin, tetrahydrocurcumin, ethyl ferulate, ethyl gallate, resveratrol, 3,5-di-tert-butyl-4-hydroxytoluene, pentaerythrityl tetra-di-t-butyl hydroxyhydrocinnamate, 2,2′-methylenebis(6-tert-butyl-4-methyl-phenol), didodecyl 3,3′-thiodipropionate (available as TINOGARD® DA), tetramethylhydroxypiperidinol (available as TINOGARD® Q), ubiquinone, ubiquinol, 4-propylsyringol, and a combination thereof.

In an embodiment, the antioxidant comprises 3,5-di-tert-butyl-4-hydroxytoluene.

In another embodiment, the stabilizing mixture does not contain 3,5-di-tert-butyl-4-hydroxytoluene.

In an embodiment, the antioxidant comprises vitamin E, didodecyl 3,3′-thiodipropionate, or a combination thereof.

The amount of the antioxidant in the stabilizing mixture is not particularly limited. However, in some embodiments, the antioxidant is present in an amount of from 0% to 50%, typically 0.01% to 30%, more typically 0.1% to 15%, by weight relative to the total amount of the stabilizing mixture. In an embodiment, the antioxidant is present in an amount of from 1% to 15%, 2% to 14%, 3% to 13%, 4% to 12%, 5% to 11%, or 9% to 11%, by weight relative to the total amount of the stabilizing mixture.

The metal chelator is any compound or combination of compounds capable of binding or sequestering metal cations, particularly those that tend to act as catalysts of oxidation. Exemplary metal chelators include, but are not limited to, phytic acid, or a salt thereof (available as DERMOFEEL® PA-12), citric acid, tetrasodium iminodisuccinate, tetrasodium glutamate diacetate, trisodium dicarboxymethyl alaninate, ethylenediaminetetraacetic acid, trans-1,2-diaminocyclohexane-N, N,N′, N′-tetraacetic acid, a poloxamine (available as TETRONIC® 1301), phosphoric acid bis(2-ethylhexyl) ester (available as WAYFOS A), and a combination thereof.

In some cases, the use of citric acid as chelator in some applications may not be preferred due to precipitation in hydroalcoholic environment.

Thus, in some embodiments, the fragrance composition is free of citric acid.

In an embodiment, the metal chelator comprises phytic acid, or a salt thereof, tetrasodium iminodisuccinate, tetrasodium glutamate diacetate, trisodium dicarboxymethyl alaninate, ethylenediaminetetraacetic acid, trans-1,2-diaminocyclohexane-N, N,N′, N′-tetraacetic acid, a poloxamine, phosphoric acid bis(2-ethylhexyl) ester, and a combination thereof.

In an embodiment, the metal chelator comprises a poloxamine, phosphoric acid bis(2-ethylhexyl) ester, phytic acid, or a salt thereof, or a combination thereof.

In an embodiment, the metal chelator comprises phytic acid, or a salt thereof. In an embodiment, the metal chelator comprises a salt of phytic acid, typically sodium or potassium salt of phytic acid.

The amount of the metal chelator in the stabilizing mixture is not particularly limited. However, in some embodiments, the metal chelator is present in an amount of from 0% to 50%, typically 0.001% to 35%, more typically 0.003% to 10%, still more typically 0.004% to 5%, by weight relative to the total amount of the stabilizing mixture. In an embodiment, the metal chelator is present in an amount of from 0.004% to 0.1%, 0.004% to 0.09%, 0.004% to 0.08%, 0.004% to 0.07%, 0.004% to 0.06%, 0.004% to 0.05%, 0.004% to 0.04%, 0.004% to 0.03%, 0.004% to 0.02%, or 0.004% to 0.01%, by weight relative to the total amount of the stabilizing mixture.

The pH regulator is any compound or combination of compounds capable of modifying and/or maintaining the pH of the stabilizing mixture, or compositions containing the said stabilizing mixture. Exemplary pH regulators include, but are not limited to, compounds selected from the group consisting of sodium, potassium, and ammonium salts of carbonate, bicarbonate, phosphate, monohydrogenphosphate, dihydrogenphosphate, lactate, citrate, succinate, and hydroxide; dimethylglucamine, methylglucamine, tetrahydroxypropyl ethylenediamine, glycine, mono- and diethyl citrate, ethyl succinate, triethanolamine, an alkyldiethanoleamine, such as methyldiethanolamine, tricine, tris buffer, and a combination thereof. The pH regulator may comprise a mixture of sodium monohydrogenphosphate and sodium dihydrogenphosphate at varying ratios to yield buffered aqueous solutions at defined pH values. As used herein, such mixtures will be referred to as phosphate buffers.

In some cases, the use of citric acid as pH regulator in some applications may not be preferred due to precipitation in hydroalcoholic environment.

In an embodiment, the pH regulator comprises triethanolamine, diethyl citrate, tetrahydroxypropyl ethylenediamine, an alkyldiethanolamine, or a combination thereof.

In another embodiment, the pH regulator comprises triethanolamine and diethyl citrate.

In yet another embodiment, the pH regulator comprises sodium bicarbonate, sodium hydroxide or a phosphate buffer.

The amount of the pH regulator in the stabilizing mixture is not particularly limited.

However, in some embodiments, the pH regulator is present in an amount of from 0% to 50%, typically 0.001% to 25%, more typically 0.01% to 5%, by weight relative to the total amount of the stabilizing mixture. In an embodiment, the pH regulator is present in an amount of from 0.01% to 2%, 0.01% to 1%, or 0.01% to 0.5%, by weight relative to the total amount of the stabilizing mixture. In an embodiment, the pH regulator is present in an amount of from 0.02% to 0.5% by weight relative to the total amount of the stabilizing mixture.

In an embodiment, the stabilizing mixture comprises at least an antioxidant and a pH regulator. In an embodiment, the stabilizing mixture comprises an antioxidant and a pH regulator, wherein the antioxidant comprises Vitamin E and the pH regulator comprises a phosphate buffer.

In another embodiment, the stabilizing mixture comprises an antioxidant, a metal chelator and a pH regulator, wherein the antioxidant comprises Vitamin E, the metal chelator comprises phytic acid sodium salt, and the pH regulator comprises a phosphate buffer.

As used herein, a POV remediant is any compound or combination of compounds capable of reducing the POV of the stabilizing mixture, or compositions comprising the said mixture.

As used herein, the term “peroxide value” or “POV” refers to the amount of equivalents of oxidizing potential per 1 kilogram of material. Without intending to be limited to any particular theory, the POV of a material is determined analytically. The term “POV” does not refer to a chemical compound or group of compounds but is often used loosely and interchangeably with the products of autoxidation within a sample that cause a response during a POV test. These autoxidation products differ depending upon the particular material being tested. Many classes of chemical compounds will produce a response during a POV test, including but not limited to organic and inorganic hydroperoxides, organic and inorganic peroxides, peroxyhemiacetals, peroxyhemiketals, and hydrogen peroxide itself.

The POV of the stabilizing mixture, or compositions comprising the stabilizing mixture, may be determined by any method readily selectable by one of ordinary skill in the art. Non-limiting examples include iodometric titration, high-performance liquid chromatography, and the like. An exemplary method for determining POV is disclosed in Calandra et al., Flavour and Fragr. J. (2015), 30, p 121-130.

The POV remediant may comprise an α-oxocarboxylic acid, or a salt thereof, or a 2-hydroxyketone.

Exemplary α-oxocarboxylic acids, or salts thereof, include, but are not limited to, sodium alpha keto glutarate, pyruvic acid, 2-oxovaleric acid, phenylglyoxylic acid, 2-oxobutyric acid, 2-oxo-2-furanacetic acid, oxaloacetic acid, α-ketoglutaric acid, 2-oxopentandioate, indole-3-pyruvic acid, 2-thiopheneglyoxylic acid, trimethylpyruvic acid, 2-oxoadipic acid, 4-hydroxyphenylpyruvic acid, phenylpyruvic acid, 2-oxooctanoic acid, and a combination thereof.

Exemplary 2-hydroxyketones include, but are not limited to, acetoin, erythrulose, dihydroxyacetone, 2-keto-D-gluconic acid hemicalcium salt hydrate, (1S,2S,5S)-(−)-2-hydroxy-3-pinanone, benzoin, p-anisoin, 2-hydroxy-3-oxo-hexadecane, and a combination thereof.

The amount of the POV remediant in the stabilizing mixture is not particularly limited. However, in some embodiments, the POV remediant is present in an amount of from 0% to 50%, typically 0.01% to 20%, more typically 0.1% to 5%, by weight relative to the total amount of the stabilizing mixture. In an embodiment, the POV remediant is present in an amount of from 0.1% to 1%, typically 0.1% to 0.5%, more typically 0.2% to 0.3%, by weight relative to the total amount of the stabilizing mixture.

The antioxidant, metal chelator, pH regulator, and/or the POV remediant may be obtained from commercial sources or prepared by synthesis methods known to those of ordinary skill in the art. The antioxidant, metal chelator, pH regulator, and/or the POV remediant may be obtained from sources of natural origin, such as renewable carbon sources.

In an embodiment, the stabilizing mixture is in the form of a solution or an emulsion, typically microemulsion, more typically water-in-oil microemulsion.

In some embodiments, the stabilizing mixtures, in the form of solutions or microemulsions, are transparent at room temperature (RT) and are stable within a large temperature range (for example RT±10-20° C., typically between 10° C. and 45° C., more typically between 3° and 45° C.), and over an extended time period, typically at least a few days, more typically at least a few weeks, even more typically at least a few months, or longer.

The term transparent means that the stabilizing mixtures in the absence of coloring or fluorescent agents have transmittance values in the visible light (500-800 nm) of 100% at a path length of 1 cm referenced against demineralized water.

The term stable means that the stabilizing mixtures do not phase separate and remain transparent without detectable signs of turbidity, cloudiness, or precipitates.

The solution typically comprises the stabilizing mixture described herein and a solvent capable of solubilizing the mixture such that the solution is transparent and homogeneous. Suitable solvents include, but are not limited to, C₁-C₆ alcohols, such as ethanol, n-propanol, 2-propanol, and 1-butanol; diols, such as 1,2-propanediol, 1,2-butanediol, 1,2-pentanediol, 1,2-hexanediol, 1,3-propanediol, 1,3-butanediol, 2,3-butanediol, and 2-methyl-2,4-pentanediol; polyalkylene glycols, such as dipropylene glycol; glycerol; isopropylidene glycerol, 1,2,3-propanetriyl triacetate, dimethyl glutarate, dimethyl adipate, 1,3-diacetyloxypropan-2-yl acetate, diethyl phthalate, isopropyl myristate, rosin resins (such as Abalyn®), benzyl benzoate, benzyl alcohol, 2-(2-ethoxyethoxy)-1-ethanol, triethyl citrate, and mixtures thereof; n-alkane, an iso-alkane, a cycloalkane, an n-alkene, an iso-alkene, a cycloalkene, an alkyl ester, a medium chain triglyceride, a silicon oil, a vegetable oil, a mineral oil, a paraffine, a perfume raw material, or a combination thereof, and naturally derived solvents, such as glycerol and vegetable oils, including palm oil, sunflower oil, linseed oil, and mixtures thereof.

In an embodiment, the stabilizing mixture is in the form of a solution in dipropylene glycol or isopropyl myristate.

In an embodiment, the stabilizing mixture does not contain dipropylene glycol.

In an embodiment, the stabilizing mixture is in the form of an emulsion, typically a microemulsion, more typically a water-in-oil microemulsion, comprising the stabilizing mixture described herein.

In an embodiment, the stabilizing mixture is the form of a water-in-oil microemulsion comprising:
a) a discontinuous aqueous phase comprising water;
b) a continuous oil phase;
c) a surfactant; and
d) a water-miscible solvent.

As understood by a person of ordinary skill in the art, an emulsion, such as a macroemulsion, a nanoemulsion, or a microemulsion, is a mixture of two liquids that are immiscible due to their different polarities (hydrophobic vs. hydrophilic). In an emulsion, one liquid (dispersed, discontinuous, or internal phase) is dispersed in another liquid (external or continuous phase). One liquid is typically polar and the other liquid is typically non-polar. Therefore, the non-polar phase may be dispersed within the polar phase, or the polar phase may be dispersed within the non-polar phase. When the non-polar phase, typically an oil phase, is dispersed within the polar phase, typically an aqueous phase, such an emulsion is an oil-in-water emulsion. When the polar phase is dispersed within the non-polar phase, the emulsion is a water-in-oil emulsion.

Double emulsions, such as water-in-oil-in-water (WOW) emulsions and its opposite, oil-in-water-in-oil (OWO) emulsions, with three distinct phases are known. With respect to the WOW type, the three distinct phases consist of polar phase droplets that are dispersed in a non-polar phase, which is then enclosed in a continuous polar phase. Such three-phase emulsions are not contemplated in the present disclosure. Therefore, the emulsions of the present disclosure are not double emulsions, i.e., the emulsions of the present disclosure are neither water-in-oil-in-water (WOW) emulsions nor oil-in-water-in-oil (OWO) emulsions.

In an embodiment, the discontinuous water phase is 0.1 to 30 wt %, the continuous oil phase is 1 to 50 wt %, and the surfactant is 10 to 90 wt %, relative to the total weight of the emulsion.

Exemplary surfactants suitable for use in the microemulsion of the present disclosure include, but are not limited to, sorbitan fatty acid ester, sorbitan monoester, sorbitan monolaurate, sorbitan monooleate, polyoxyethylene sorbitan fatty acid ester, polyoxyethylene sorbitan monoester, polyoxyethylene (20) sorbitan monolaurate, polyoxyethylene (20) sorbitan monooleate; polyglyceryl fatty acid esters, glyceryl monooleate, polyglyceryl-3 caprate, polyglyceryl-4 caprate, polyglyceryl-6 caprylate, polyglyceryl-3 cocoate, polyglyceryl-6 ricinoleate, polyglyceryl-10 laurate; a C₅-C₂₂ alkyl polyglucoside, a C₆-C₂₂ alkyl xyloside, fatty acids, lecithin, phosphatidylcholine, lysolecithin, natural and modified biosurfactants, such as saponins and glycolipids, for example, sophorolipids, mannosylerythritollipids and rhamnolipids; mixed propoxylated and ethoxylated aliphatic alcohols, linear and branched, primary and secondary alcohols with a chain length containing a total number of carbon atoms between 8 and 18 modified with a polyoxyethylene group carrying between 1 and 40 PEG units, PEG-modified hydrogenated castor oil carrying between 20 and 60 PEG units, triblock copolymers comprising blocks of polyethyleneglycol and polypropyleneglycol, and a combination thereof.

In an embodiment, the surfactant is selected from the group consisting of sorbitan monolaurate and polyoxyethylene (20) sorbitan monolaurate, and combinations thereof.

In another embodiment, the surfactant is sorbitan monolaurate.

In an embodiment, the continuous oil phase comprises an n-alkane, an iso-alkane, a cycloalkane, an n-alkene, an iso-alkene, a cycloalkene, an alkyl ester, a medium chain triglyceride, a silicon oil, a vegetable oil, a mineral oil, a paraffine, a perfume raw material, or a combination thereof. In an embodiment, the continuous oil phase comprises undecane and tridecane (available as Cetiol® Ultimate).

In an embodiment, the discontinuous water phase comprises the pH regulator, wherein the pH regulator is water-soluble.

Suitable pH regulators for use in the discontinuous water phase are those described herein that are water-soluble. Examples of water-soluble pH regulators include, but are not limited to, sodium, potassium, or ammonium salts of carbonate, bicarbonate, phosphate, monohydrogenphosphate, dihydrogenphosphate, lactate, citrate, succinate, and hydroxide; dimethylglucamine, methylglucamine, tetrahydroxypropyl ethylenediamine, glycine, mono- and diethyl citrate, ethyl succinate, triethanolamine, tricine, tris buffer, and combinations thereof.

In an embodiment, the discontinuous water phase comprises the antioxidant and/or POV remediant, wherein the antioxidant and/or POV remediant are/is water-soluble.

Suitable antioxidants for use in the discontinuous water phase are those described herein that are water-soluble. Examples of water-soluble antioxidants include, but are not limited to, ascorbic acid, a natural extract, such as rosemary extract, gallic acid, ferulic acid, caffeic acid, tannic acid, chlorogenic acid, ellagic acid, rosmarinic acid, a polyphenol, tetramethylhydroxypiperidinol (available as Tinogard® Q), and combinations thereof.

Exemplary water-soluble POV remediants include, but are not limited to, alkali metal and ammonium salts of α-ketoglutaric acid, typically sodium α-ketoglutarate.

In an embodiment, the discontinuous water phase comprises the metal chelator, wherein the metal chelator is water-soluble.

Exemplary water-soluble metal chelators include, but are not limited to, phytic acid, or a salt thereof.

In an embodiment, the continuous oil phase comprises the antioxidant and/or the POV remediant, wherein the antioxidant and/or the POV remediant are/is oil-soluble.

Suitable antioxidants for use in the continuous oil phase are those described herein that are oil soluble. Examples of oil-soluble antioxidants include, but are not limited to, vitamin E, α-tocopherol, β-tocopherol, γ-tocopherol, δ-tocopherol, ascorbyl palmitate, natural extracts, such as rosemary extract, curcumin, tetrahydrocurcumin, ethyl ferulate, ethyl gallate, resveratrol, 3,5-di-tert-butyl-4-hydroxytoluene, pentaerythrityl tetra-di-t-butyl hydroxyhydrocinnamate, 2,2′-methylenebis(6-tert-butyl-4-methyl-phenol), didodecyl 3,3′-thiodipropionate, ubiquinone, ubiquinol, 4-propyl syringol, and combinations thereof.

Suitable POV remediants for use in the continuous oil phase are those described herein that are oil soluble. Examples of oil-soluble POV remediants include, but are not limited to, alkyl amine salts of α-ketoglutaric acid, such as dimethyldodecylammonium α-ketoglutarate (dimethyldodecylamine salt of α-ketoglutaric acid).

The water-miscible solvent may be any organic solvent or blend of organic solvents that is miscible with water. Suitable water-miscible solvents include, but are not limited to, C₁-C₆ alcohols, such as ethanol, n-propanol, 2-propanol, and 1-butanol; diols, such as 1,2-propanediol, 1,2-butanediol, 1,3-butanediol, 2,3-butanediol, 1,2-pentanediol (available as A-Leen® 5), 1,2-hexanediol, 1,3-propanediol (available as Zemea® Propanediol), and 2-methyl-2,4-pentanediol; polyalkylene glycols, such as dipropylene glycol; polyols, such as glycerol; triethyl citrate, isopropylidene glycerol, ethyl lactate, and combinations thereof.

The fragrance oil of the fragrance composition comprises one or more perfumery ingredients. By a “perfuming ingredient”, it is meant here a compound of current use in perfumery, which is used essentially for its ability to smell pleasantly and to be capable of imparting a pleasant odor to the products into which it is incorporated, or to the surfaces, such as skin or hair, to which it is applied, on its own or in admixture with other such ingredients. Perfuming ingredients are well-known to those of ordinary skill in the art. The nature and type of the perfuming ingredients do not warrant a more detailed description here, which in any case would not be exhaustive, the person of ordinary skill in the art being able to select them on the basis of his/her general knowledge and according to the intended use or application and the desired organoleptic effect. In general terms, perfuming ingredients belong to chemical classes as varied as alcohols, lactones, aldehydes, ketones, esters, ethers, acetates, nitriles, terpenoids, nitrogenous or sulphurous heterocyclic compounds, and essential oils. Perfuming ingredients can be of natural or synthetic origin. Suitable perfumery ingredients are in any case listed in reference texts such as the book by S. Arctander, Perfume and Flavor Chemicals, 1969, Montclair, New Jersey, USA, or its more recent versions, or in other works of a similar nature, as well as in the abundant patent literature in the field of perfumery. It is also understood that said perfuming ingredients may include compounds known to release, in a controlled manner, various types of perfuming compounds, known as properfume or profragrance.

The amount of fragrance oil in the fragrance composition is not particularly limited. However, in some embodiments, the fragrance oil is present in an amount of from 50% to 99%, typically 80% to 99%, more typically 90% to 99%. In some embodiments, the fragrance oil is present in an amount of from 95% to 99%.

In a particular embodiment, the fragrance oil is present in an amount of 98%.

In another embodiment, the fragrance oil is present in an amount of 99%.

The fragrance composition may further comprise a perfuming co-ingredient. As used herein, a perfuming co-ingredient refers to ingredients that impart a hedonic effect, i.e., used for the primary purpose of conferring or modulating an odor. In other words, such a co-ingredient, to be considered as being a perfuming one, must be recognized by a person with skill in the art as being able to impart or modify in a positive or pleasant way the odor of a composition, and not just as having an odor. Perfuming co-ingredients may impart an additional benefit beyond that of modifying or imparting an odor, such as long-lasting, blooming, malodour counteraction, antimicrobial effect, antiviral effect, microbial stability, or pest control.

The fragrance composition may further comprise additional ingredients typically used in the perfumery industry, such as a perfumery carrier.

As used herein, “perfumery carrier” refers to a material which is practically neutral from a perfumery point of view, i.e., that it does not significantly alter the organoleptic properties of perfuming ingredients. Said carrier may be a liquid or a solid. Exemplary liquid carriers include, but are not limited to, an emulsifying system, i.e., a solvent and a surfactant system, or a solvent typically used in perfumery. A detailed description of the nature and type of solvents commonly used in perfumery cannot be exhaustive. However, in some instances, for example, the perfumery carrier may be ethanol, water/ethanol mixtures, limonene or other terpenes, isoparaffins such as those known under the trademark Isopar® (available from Exxon Chemical) or glycol ethers and glycol ether esters, such as those known under the trademark Dowanol® (available from Dow Chemical Company), or hydrogenated castors oils such as those known under the trademark Cremophor® RH 40 (available from BASF). Other exemplary solvents suitable for use as perfumery carriers include, but are not limited to, glycerol, dipropylene glycol and its monoether, 1,2,3-propanetriyl triacetate, dimethyl glutarate, dimethyl adipate 1,3-diacetyloxypropan-2-yl acetate, diethyl phthalate, isopropyl myristate, Abalyn® (rosin resins), benzyl benzoate, benzyl alcohol, 2-(2-ethoxyethoxy)-1-ethanol, triethyl citrate or mixtures thereof. Naturally derived solvents, like glycerol or various vegetable oils such as palm oil, sunflower oil or linseed oil, may also be used.

Solid carriers are materials to which the fragrance composition or some element of the fragrance composition can be chemically or physically bound. In general, such solid carriers are employed either to stabilize the composition, or to control the rate of evaporation of the compositions or of some ingredients. Solid carriers are of current use in the art and a person skilled in the art knows how to reach the desired effect. Suitable solid carriers include, but are not limited to, absorbing gums or polymers or inorganic materials, such as porous polymers, cyclodextrines, dextrines, maltodextrines wood-based materials, organic or inorganic gels, clays, gypsum talc or zeolites.

Other suitable solid carriers include encapsulating materials. Examples of such materials may comprise wall-forming and plasticizing materials, such as glucose syrups, natural or modified starches, hydrocolloids, cellulose derivatives, polyvinyl acetates, polyvinylalcohols, proteins or pectins, plant gums such as acacia gum (Gum Arabic), urea, sodium chloride, sodium sulphate, zeolite, sodium carbonate, sodium bicarbonate, clay, talc, calcium carbonate, magnesium sulfate, gypsum, calcium sulfate, magnesium oxide, zinc oxide, titanium dioxide, calcium chloride, potassium chloride, magnesium chloride, zinc chloride, carbohydrates, saccharides such as sucrose, mono-, di-, and polysaccharides and derivatives such as chitosan, starch, cellulose, carboxymethyl methylcellulose, methylcellulose, hydroxyethyl cellulose, ethyl cellulose, propyl cellulose, polyols/sugar alcohols such as sorbitol, maltitol, xylitol, erythritol, and isomalt, polyethylene glycol (PEG), polyvinyl pyrrolidin (PVP), polyvinyl alcohol, acrylamides, acrylates, polyacrylic acid and related, maleic anhydride copolymers, amine-functional polymers, vinyl ethers, styrenes, polystyrenesulfonates, vinyl acids, ethylene glycol-propylene glycol block copolymers, vegetable gums, gum acacia, pectins, xanthanes, alginates, carragenans, or any water soluble solid acid, fatty alcohols or fatty acids and mixtures thereof.

Other suitable encapsulating materials are described in reference texts known to those of skill in the art, such as H. Scherz, Hydrokolloide: Stabilisatoren, Dickungs-und Geliermittel in Lebensmitteln, Band 2 der Schriftenreihe Lebensmittelchemie, Lebensmittelqualität, Behr's Verlag Gmbh & Co., Hamburg, 1996. The encapsulation is a well-known process to a person skilled in the art, and may be performed, for instance, by using techniques such as spray-drying, agglomeration or yet extrusion; or consists of a coating encapsulation, including coacervation and complex coacervation techniques.

Other exemplary solid carriers include core-shell capsules with resins of aminoplast, polyamide, polyester, polyurea or polyurethane type, and mixtures threof, made using techniques well-known to those of ordinary skill in the art, such as phase separation induced by polymerization, interfacial polymerization, coacervation, or a combination thereof, optionally in the presence of a polymeric stabilizer or of a cationic copolymer.

Resins may be produced by the polycondensation of an aldehyde (e.g., formaldehyde, 2,2-dimethoxyethanal, glyoxal, glyoxylic acid or glycolaldehyde, and mixtures thereof) with an amine such as urea, benzoguanamine, glycoluryl, melamine, methylol melamine, methylated methylol melamine, guanazole and the like, as well as mixtures thereof. Alternatively, one may use preformed resins like alkylolated polyamines such as those commercially available under the trademark Urac® (origin: Cytec Technology Corp.), Cymel® (origin: Cytec Technology Corp.), Urecoll® or Luracoll® (origin: BASF).

Other suitable resins are the those produced by the polycondensation of a polyol, like glycerol, and a polyisocyanate, for example, a trimer of hexamethylene diisocyanate, a trimer of isophorone diisocyanate or xylylene diisocyanate or a Biuret of hexamethylene diisocyanate or a trimer of xylylene diisocyanate with trimethylolpropane (marketed as Takenate® by Mitsui Chemicals), among which a trimer of xylylene diisocyanate with trimethylolpropane and a Biuret of hexamethylene diisocyanate are of mention.

The encapsulation of perfumes by polycondensation of amino resins, namely melamine-based resins with aldehydes is well-known in the art. Pertinent publications include, but are not limited to, K. Dietrich et al. Acta Polymerica, 1989, vol. 40, pages 243, 325 and 683, as well as 1990, vol. 41, page 91 and U.S. Pat. No. 4,396,670 issued Aug. 2, 1983. The general knowledge in encapsulation technology is very significant and cannot be exhaustive. More recent publications of pertinence, which disclose suitable uses of such microcapsules, are represented, for example, by the article of K. Bruyninckx and M. Dusselier, ACS Sustainable Chemistry & Engineering, 2019, vol. 7, pages 8041-8054. These publications are incorporated herein by reference.

The fragrance composition may optionally comprise at least one perfumery adjuvant.

The at least one perfumery adjuvant is an ingredient capable of imparting an additional added benefit such as a color, a particular light resistance, etc. A detailed description of the nature and type of adjuvant commonly used in perfuming compositions cannot be exhaustive, but mention is made that said ingredients are well known to a person skilled in the art.

Exemplary perfumery adjuvants include, but are not limited to, viscosity agents (e.g., surfactants, thickeners, gelling and/or rheology modifiers), coloring agents (e.g., dyes and/or pigments), preservatives (e.g. antibacterial or antimicrobial or antifungal or anti irritant agents), abrasives, skin cooling agents, fixatives, insect repellants, ointments, nutrient, and mixtures thereof.

Fixatives, also known as “modulators”, are agents having the capacity to affect the manner in which the odor, and in particular the evaporation rate and intensity, of the compositions incorporating said modulator can be perceived by an observer or user thereof, over time, as compared to the same perception in the absence of the modulator. In particular, the modulator allows prolonging the time during which their fragrance is perceived. Suitable modulators include, but are not limited to, methyl glucoside polyol; ethyl glucoside polyol; propyl glucoside polyol; isocetyl alcohol; PPG-3 myristyl ether; neopentyl glycol diethylhexanoate; sucrose laurate; sucrose dilaurate, sucrose myristate, sucrose palmitate, sucrose stearate, sucrose distearate, sucrose tristearate, hyaluronic acid disaccharide sodium salt, sodium hyaluronate, propylene glycol propyl ether; dicetyl ether; polyglycerin-4 ethers; isoceteth-5; isoceteth-7, isoceteth-10; isoceteth-12; isoceteth-15; isoceteth-20; isoceteth-25; isoceteth-30; disodium lauroamphodipropionate; hexaethylene glycol monododecyl ether; and their mixtures; neopentyl glycol diisononanoate; cetearyl ethylhexanoate; panthenol ethyl ether, DL-panthenol, N-hexadecyl n-nonanoate, noctadecyl n-nonanoate, a profragrance, cyclodextrin, an encapsulation, and any combination thereof.

The fragrance composition may optionally comprise UV filters. Exemplary UV filters include, but are not limited to, butyl methoxydibenzoylmethane, octocrylene, 4-methylbenzylidene camphor, ethylhexyl salicylate, homosalate, phenylbenzimidazole sulfonic acid, methylene bis-benzotriazolyl tetramethylbutylphenol, ethylhexyl methoxycinnamate, bis-ethylhexyloxyphenol methoxyphenyl triazine, and the like.

The amount of the stabilizing mixture, solution, or emulsion of the present disclosure in the fragrance composition is not particularly limited. However, in some embodiments, the stabilizing mixture, solution, or emulsion is present in an amount from 0.01% to 5%, typically from 0.02% to 2%, more typically from 1 to 2%, by weight relative to the total weight of the fragrance composition

In an embodiment, the stabilizing mixture is miscible with the fragrance oil.

The fragrance compositions according to the present disclosure are transparent at room temperature (RT) and are stable within a large temperature range (for example RT+10-20° C., typically between 10° C. and 45° C., more typically between 3° and 45° C.) and over an extended time period. In some embodiments, the fragrance compositions according to the present disclosure are stable at temperatures of from 3° C. to 45° C., typically from 25° C. to 45° C.

The term transparent means that the fragrance compositions in the absence of coloring or fluorescent agents have transmittance values in the visible light (500-800 nm) of 100% at a path length of 1 cm referenced against demineralized water.

The term stable means that the fragrance compositions do not phase separate and remain transparent without detectable signs of turbidity, cloudiness, or precipitates.

The fragrance composition contains less than about 1 wt % water, relative to the total weight of the composition. In some embodiments, the composition comprises less than or equal to about 0.9 wt %, less than or equal to about 0.8 wt %, less than or equal to about 0.7 wt %, less than or equal to about 0.6 wt %, less than or equal to about 0.5 wt %, less than or equal to about 0.4 wt %, less than or equal to about 0.3 wt %, less than or equal to about 0.2 wt % water, less than or equal to about 0.1 wt %, or less than or equal to about 0.05 wt % water, relative to the total weight of the composition.

The fragrance compositions described herein may be incorporated in consumer products, as will be described below. Thus, in an embodiment, the consumer product according to the present disclosure is transparent and stable at RT.

The fragrance compositions according to the present disclosure may be prepared according to any method known to those of ordinary skill in the art. The ordinarily skilled artisan is perfectly able to design optimal formulations for the desired effect by admixing the above-mentioned components to arrive at the desired composition by applying standard knowledge and concepts known to those of ordinary skill and by utilizing routine optimization methodologies.

In the second aspect, the present disclosure relates to a solution comprising:
one or more solvents, and
at least 2 ingredients, typically at least 3 ingredients, each having a function selected from the group consisting of antioxidant, metal chelator, pH regulator, and POV remediant,
wherein the at least 2 ingredients, typically at least 3 ingredients, do not have the same function.

The features and limitations of the stabilizing mixture described herein apply, mutatis mutandis, to the solution.

The one or more solvents is/are selected from solvents that include, but are not limited to, C₁-C₆ alcohols, such as ethanol, n-propanol, 2-propanol, and 1-butanol; diols, such as 1,2-propanediol, 1,2-butanediol, 1,2-pentanediol, 1,2-hexanediol, 1,3-propanediol, 1,3-butanediol, 2,3-butanediol, and 2-methyl-2,4-pentanediol; polyalkylene glycols, such as dipropylene glycol; glycerol; isopropylidene glycerol, 1,2,3-propanetriyl triacetate, dimethyl glutarate, dimethyl adipate, 1,3-diacetyloxypropan-2-yl acetate, diethyl phthalate, isopropyl myristate, rosin resins (such as Abalyn®), benzyl benzoate, benzyl alcohol, 2-(2-ethoxyethoxy)-1-ethanol, triethyl citrate, and mixtures thereof; n-alkane, an iso-alkane, a cycloalkane, an n-alkene, an iso-alkene, a cycloalkene, an alkyl ester, a medium chain triglyceride, a silicon oil, a vegetable oil, a mineral oil, a paraffine, a perfume raw material, or a combination thereof, and naturally derived solvents, such as glycerol and vegetable oils, including palm oil, sunflower oil, linseed oil, and mixtures thereof.

The solution described herein may be made according to methods known to those of ordinary skill in the art. One exemplary method is combining the one or more solvents, the at least 2 ingredients, each having a function selected from the group consisting of antioxidant, metal chelator, pH regulator, and POV remediant, followed by mixing until a homogeneous solution is obtained.

In the third aspect, the present disclosure relates to a water-in-oil microemulsion comprising:
a) a discontinuous aqueous phase comprising water and one or more water-soluble compounds each having a function selected from the group consisting of antioxidant, metal chelator, pH regulator, POV remediant, and combinations thereof;
b) a continuous oil phase comprising one or more oil-soluble compounds each having a function selected from the group consisting of antioxidant, metal chelator, pH regulator, POV remediant, and combinations thereof;
c) a surfactant; and
d) a water-miscible solvent.

The features and limitations of the stabilizing mixture described herein apply, mutatis mutandis, to the water-in-oil microemulsion.

The one or more water-soluble compounds selected from the group consisting of a pH regulator, an antioxidant, a chelator, a POV remediant, and combinations thereof, and the one or more oil-soluble compounds selected from the group consisting of a pH regulator, an antioxidant, a chelator, a POV remediant, and combinations thereof are as described herein.

Analogously, suitable surfactants and water-miscible solvents are as described herein.

In an embodiment, the surfactant is selected from the group consisting of sorbitan monolaurate and polyoxyethylene (20) sorbitan monolaurate, and mixtures thereof. In another embodiment, the surfactant is sorbitan monolaurate.

The water-in-oil microemulsions described herein may be made according to methods known to those of ordinary skill in th art. In an exemplary method, the aqueous phase comprising water and one or more water-soluble compounds each having a function selected from the group consisting of antioxidant, metal chelator, pH regulator, POV remediant, and combinations thereof is prepared by mixing the components. Separately, the oil phase comprising one or more oil-soluble compounds each having a function selected from the group consisting of antioxidant, metal chelator, pH regulator, POV remediant, and combinations thereof; surfactant, and water-miscible solvent are mixed. Subsequently, the aqueous phase and the oil/surfactant/co-solvent phases are mixed.

In the fourth aspect, the present disclosure relates to a consumer product comprising the fragrance composition described herein or the water-in-oil microemulsion described herein.

The form of the consumer product is not particularly limited. In an embodiment, the consumer product is a perfume, typically a fine fragrance, a fabric care product, a body-care product, a cosmetic preparation, a skin-care product, an air care product, or a home care product.

In another embodiment, the consumer product is an eau de toilette, a hydro-alcoholic solution, an ethanol free eau de toilette, a splash, eau de parfum, a cologne, a shave or after-shave lotion, a liquid or solid or unit-dose detergent, a fabric softener, a solid or liquid fabric scent-booster, a fabric refresher, an ironing water, a paper, a bleach, a carpet cleaners, curtain-care products, a shampoo, a leave-on or rinse-off hair conditioner, a coloring preparation, a color care product, a hair shaping product, a dental care product, a disinfectant, an intimate care product, a hair spray, a vanishing cream, a deodorant or antiperspirant, hair remover, tanning or sun product, a nail product, a skin cleansing, a makeup, a perfumed soap, a shower or bath mousse, oil or gel, or a foot/hand care products, a hygiene product, an air freshener, a “ready to use” powdered air freshener, a mold remover, a furniture care product, a wipe, a dish detergent or hard-surface detergent, a leather care product, or a car care product.

The amount of the stabilizing mixture, solution, or emulsion of the present disclosure in the consumer product is not particularly limited. However, in some embodiments, the stabilizing mixture, solution, or emulsion is present in an amount from 0.01% to 5%, typically from 0.02% to 2%, more typically from 1 to 2%, by weight relative to the total weight of the consumer product.

In some embodiments, the consumer product contains less than about 1 wt % water, relative to the total weight of the consumer product.

In some embodiments, the consumer products according to the present disclosure are transparent at room temperature (RT) and are stable within a large temperature range (for example RT±10-20° C., typically between 10° C. and 45° C., more typically between 3° and 45° C.), and over an extended time period, typically at least a few days, more typically at least a few weeks, even more typically at least a few months, or longer.

The term transparent means that the consumer products in the absence of coloring or fluorescent agents have transmittance values in the visible light (500-800 nm) of 100% at a path length of 1 cm referenced against demineralized water.

The term stable means that the consumer products do not phase separate and remain transparent without detectable signs of turbidity, cloudiness, or precipitates.

In the fifth aspect, the present disclosure relates to use of the stabilizing mixture defined herein, the solution described herein, or the water-in-oil microemulsion described herein, to enhance the oxidative stability of a fragrance oil in a fragrance composition or a consumer product.

### Embodiments

Embodiment 1. A fragrance composition comprising:
a) a fragrance oil, and
b) a stabilizing mixture comprising at least 2 ingredients, typically at least 3 ingredients, each having a function selected from the group consisting of antioxidant, metal chelator, pH regulator, and POV remediant,
wherein the at least 2 ingredients, typically at least 3 ingredients, do not have the same function, and
wherein the composition contains less than about 1 wt % water, relative to the total weight of the composition.

Embodiment 2. The fragrance composition of embodiment 1, wherein the stabilizing mixture comprises at least 3 ingredients, each having a function selected from the group consisting of antioxidant, metal chelator, pH regulator, and POV remediant.

Embodiment 3. The fragrance composition of embodiment 1, wherein the stabilizing mixture comprises at least one antioxidant and at least one metal chelator.

Embodiment 4. The fragrance composition of embodiment 1 or 2, wherein the stabilizing mixture comprises two antioxidants and two metal chelators.

Embodiment 5. The fragrance composition of embodiment 1 or 2, wherein the stabilizing mixture comprises at least one antioxidant, at least one metal chelator, and at least one pH regulator.

Embodiment 6. The fragrance composition of embodiment 1 or 2, wherein the stabilizing mixture comprises one antioxidant, two metal chelators, and two pH regulators.

Embodiment 7. The fragrance composition of embodiment 1 or 2, wherein the stabilizing mixture comprises at least one antioxidant, at least one pH regulator, and at least one POV remediant.

Embodiment 8. The fragrance composition of embodiment 1 or 2, wherein the stabilizing mixture comprises at least one antioxidant, at least one metal chelator, at least one pH regulator, and at least one POV remediant.

Embodiment 9. The fragrance composition according to any one of embodiments 1-8, wherein the antioxidant is selected from the group consisting of ascorbic acid, gallic acid, ferulic acid, caffeic acid, tannic acid, chlorogenic acid, ellagic acid, rosmarinic acid, a polyphenol, tetramethylhydroxypiperidinol, vitamin E, α-tocopherol, β-tocopherol, γ-tocopherol, δ-tocopherol, ascorbyl palmitate, ascorbyl glucoside, natural extracts, typically rosemary extract, curcumin, tetrahydrocurcumin, ethyl ferulate, ethyl gallate, resveratrol, 3,5-di-tert-butyl-4-hydroxytoluene, pentaerythrityl tetra-di-t-butyl hydroxyhydrocinnamate, 2,2′-methylenebis(6-tert-butyl-4-methyl-phenol), didodecyl 3,3′-thiodipropionate, tetramethylhydroxypiperidinol, ubiquinone, ubiquinol, 4-propylsyringol, and a combination thereof.

Embodiment 10. The fragrance composition according to any one of embodiments 1 to 9, wherein the antioxidant comprises 3,5-di-tert-butyl-4-hydroxytoluene.

Embodiment 11. The fragrance composition according to any one of embodiments 1 to 10, wherein the stabilizing mixture does not contain 3,5-di-tert-butyl-4-hydroxytoluene.

Embodiment 12. The fragrance composition according to any one of embodiments 1 to 11, wherein the antioxidant comprises vitamin E, didodecyl 3,3′-thiodipropionate, or a combination thereof.

Embodiment 13. The fragrance composition according to any one of embodiments 1 to 12, wherein the metal chelator is selected from the group consisting of phytic acid, or a salt thereof, citric acid, tetrasodium iminodisuccinate, tetrasodium glutamate diacetate, trisodium dicarboxymethyl alaninate, ethylenediaminetetraacetic acid, trans-1,2-diaminocyclohexane-N,N,N′,N′-tetraacetic acid, a poloxamine, phosphoric acid bis(2-ethylhexyl) ester, and a combination thereof.

Embodiment 13a. The fragrance composition according to any one of embodiments 1 to 13, wherein the metal chelator is selected from the group consisting of phytic acid, or a salt thereof, tetrasodium iminodisuccinate, tetrasodium glutamate diacetate, trisodium dicarboxymethyl alaninate, ethylenediaminetetraacetic acid, trans-1,2-diaminocyclohexane-N,N,N′,N′-tetraacetic acid, a poloxamine, phosphoric acid bis(2-ethylhexyl) ester, and a combination thereof.

Embodiment 14. The fragrance composition according to any one of embodiments 1 to 13, wherein the metal chelator comprises a poloxamine, phosphoric acid bis(2-ethylhexyl) ester, phytic acid, or a salt thereof, or a combination thereof.

Embodiment 15. The fragrance composition according to any one of embodiments 1 to 14, wherein the metal chelator comprises phytic acid, or a salt thereof.

Embodiment 16. The fragrance composition according to any one of embodiments 1 to 15, wherein the pH regulator is selected from the group consisting of sodium, potassium, and ammonium salts of carbonate, bicarbonate, phosphate, monohydrogenphosphate, dihydrogenphosphate, lactate, citrate, succinate, and hydroxide; dimethylglucamine, methylglucamine, tetrahydroxypropyl ethylenediamine, glycine, mono- and diethyl citrate, ethyl succinate, triethanolamine, an alkyldiethanoleamine, such as methyldiethanolamine, tricine, tris buffer, and a combination thereof.

Embodiment 17. The fragrance composition according to any one of embodiments 1 to 16, wherein the pH regulator comprises triethanolamine, diethyl citrate, tetrahydroxypropyl ethylenediamine, an alkyldiethanolamine, or a combination thereof.

Embodiment 18. The fragrance composition according to any one of embodiments 1 to 17, wherein the pH regulator comprises triethanolamine and diethyl citrate.

Embodiment 19. The fragrance composition according to any one of embodiments 1 to 18, wherein the pH regulator comprises sodium bicarbonate, sodium hydroxide, or a phosphate buffer.

Embodiment 20. The fragrance composition according to any one of embodiments 1 to 19, wherein the stabilizing mixture comprises an antioxidant, a metal chelator and a pH regulator, wherein the antioxidant comprises Vitamin E, the metal chelator comprises phytic acid sodium salt, and the pH regulator comprises a phosphate buffer.

Embodiment 21. The fragrance composition according to any one of embodiments 1 to 20, wherein the POV remediant comprises an α-oxocarboxylic acid, or a salt thereof, or a 2-hydroxyketone.

Embodiment 22. The fragrance composition according to embodiment 21, wherein the α-oxocarboxylic acid, or a salt thereof, is selected from the group consisting of sodium alpha keto glutarate, pyruvic acid, 2-oxovaleric acid, phenylglyoxylic acid, 2-oxobutyric acid, 2-oxo-2-furanacetic acid, oxaloacetic acid, α-ketoglutaric acid, 2-oxopentandioate, indole-3-pyruvic acid, 2-thiopheneglyoxylic acid, trimethylpyruvic acid, 2-oxoadipic acid, 4-hydroxyphenylpyruvic acid, phenylpyruvic acid, 2-oxooctanoic acid, and a combination thereof.

Embodiment 23. The fragrance composition according to embodiment 21, wherein the 2-hydroxyketone is selected from the group consisting of acetoin, erythrulose, dihydroxyacetone, 2-keto-D-gluconic acid hemicalcium salt hydrate, (1S,2S,5S)-(−)-2-hydroxy-3-pinanone, benzoin, p-anisoin, benzil, 2-hydroxy-3-oxo-hexadecane, and a combination thereof.

Embodiment 24. The fragrance composition according to any one of embodiments 1 to 23, wherein the stabilizing mixture is in the form of a solution or an emulsion, typically microemulsion, more typically water-in-oil microemulsion.

Embodiment 25. The fragrance composition according to embodiment 24, wherein the stabilizing mixture is in the form of a solution in dipropylene glycol or isopropyl myristate.

Embodiment 25a. The fragrance composition according to embodiment 24, wherein the stabilizing mixture is in the form of a solution solvents chosen from C₁-C₆ alcohols, such as ethanol, n-propanol, 2-propanol, and 1-butanol; diols, such as 1,2-propanediol, 1,2-butanediol, 1,2-pentanediol, 1,2-hexanediol, 1,3-propanediol, 1,3-butanediol, 2,3-butanediol, and 2-methyl-2,4-pentanediol; polyalkylene glycols, such as dipropylene glycol; glycerol; isopropylidene glycerol, 1,2,3-propanetriyl triacetate, dimethyl glutarate, dimethyl adipate, 1,3-diacetyloxypropan-2-yl acetate, diethyl phthalate, isopropyl myristate, rosin resins (such as Abalyn®), benzyl benzoate, benzyl alcohol, 2-(2-ethoxyethoxy)-1-ethanol, triethyl citrate, and mixtures thereof; n-alkane, an iso-alkane, a cycloalkane, an n-alkene, an iso-alkene, a cycloalkene, an alkyl ester, a medium chain triglyceride, a silicon oil, a vegetable oil, a mineral oil, a paraffine, a perfume raw material, or a combination thereof, and naturally derived solvents, such as glycerol and vegetable oils, including palm oil, sunflower oil, linseed oil, and mixtures thereof.

Embodiment 26. The fragrance composition according to any one of embodiments 1 to 25, wherein the stabilizing mixture does not contain dipropylene glycol.

Embodiment 27. The fragrance composition according to embodiment 24, wherein the stabilizing mixture is the form of a water-in-oil microemulsion comprising:
a) a discontinuous aqueous phase comprising water;
b) a continuous oil phase;
c) a surfactant; and
d) a water-miscible solvent.

Embodiment 28. The fragrance composition according to embodiment 27, wherein the discontinuous water phase is 0.1 to 30 wt %, the continuous oil phase is 1 to 50 wt %, and the surfactant is 10 to 90 wt %, relative to the total weight of the microemulsion.

Embodiment 29. The fragrance composition according to any one of embodiments 27 or 28, wherein the surfactant is selected from the group consisting of sorbitan fatty acid ester, sorbitan monoester, sorbitan monolaurate, sorbitan monooleate, polyoxyethylene sorbitan fatty acid ester, polyoxyethylene sorbitan monoester, polyoxyethylene (20) sorbitan monolaurate, polyoxyethylene (20) sorbitan monooleate; polyglyceryl fatty acid esters, glyceryl monooleate, polyglyceryl-3 caprate, polyglyceryl-4 caprate, polyglyceryl-6 caprylate, polyglyceryl-3 cocoate, polyglyceryl-6 ricinoleate, polyglyceryl-10 laurate; a C₆-C₂₂ alkyl polyglucoside, a C₆-C₂₂ alkyl xyloside, fatty acids, lecithin, phosphatidylcholine, lysolecithin, natural and modified biosurfactants, such as saponins and glycolipids, for example, sophorolipids, mannosylerythritollipids and rhamnolipids; mixed propoxylated and ethoxylated aliphatic alcohols, linear and branched, primary and secondary alcohols with a chain length containing a total number of carbon atoms between 8 and 18 modified with a polyoxyethylene group carrying between 1 and 40 PEG units, PEG-modified hydrogenated castor oil carrying between 20 and 60 PEG units, triblock copolymers comprising blocks of polyethyleneglycol and polypropyleneglycol, and a combination thereof.

Embodiment 30. The fragrance composition according to any one of embodiments 27 to 29, wherein the surfactant is selected from the group consisting of sorbitan monolaurate and polyoxyethylene (20) sorbitan monolaurate, and combinations thereof.

Embodiment 31. The fragrance composition according to any one of embodiments 27 to 30, wherein the surfactant is sorbitan monolaurate.

Embodiment 32. The fragrance composition according to any one of embodiments 27 to 31, wherein the continuous oil phase comprises an n-alkane, an iso-alkane, a cycloalkane, an n-alkene, an iso-alkene, a cycloalkene, an alkyl ester, a medium chain triglyceride, a silicon oil, a vegetable oil, a mineral oil, a paraffine, a perfume raw material, or a combination thereof.

Embodiment 33. The fragrance composition according to any one of embodiments 27 to 32, wherein the discontinuous water phase comprises the pH regulator, wherein the pH regulator is water-soluble.

Embodiment 34. The fragrance composition according to any one of embodiments 27 to 33, wherein the discontinuous water phase comprises the antioxidant and/or POV remediant, wherein the antioxidant and/or POV remediant are/is water-soluble.

Embodiment 35. The fragrance composition according to any one of embodiments 27 to 34, wherein the discontinuous water phase comprises the metal chelator, wherein the metal chelator is water-soluble.

Embodiment 36. The fragrance composition according to any one of embodiments 27 to 35, wherein the continuous oil phase comprises the antioxidant and/or POV remediant, wherein the antioxidant and/or POV remediant are/is oil-soluble.

Embodiment 37. The fragrance composition according to any one of embodiments 27 to 36, wherein the water-miscible solvent is selected from the group consisting of C₁-C₆ alcohols, such as ethanol, n-propanol, 2-propanol, and 1-butanol; diols, such as 1,2-propanediol, 1,2-butanediol, 1,2-pentanediol, 1,2-hexanediol, 1,3-propanediol, 1,3-butanediol, 2,3-butanediol, and 2-methyl-2,4-pentanediol; polyalkylene glycols, such as dipropylene glycol; polyols, such as glycerol; triethyl citrate, isopropylidene glycerol, ethyl lactate, and combinations thereof.

Embodiment 38. A solution comprising:
one or more solvents, and
at least 2 ingredients, typically at least 3 ingredients, each having a function selected from the group consisting of antioxidant, metal chelator, pH regulator, and POV remediant,
wherein the at least 2 ingredients, typically at least 3 ingredients, do not have the same function.

Embodiment 39. A water-in-oil microemulsion comprising:
a) a discontinuous aqueous phase comprising water and one or more water-soluble compounds each having a function selected from the group consisting of antioxidant, metal chelator, pH regulator, POV remediant, and combinations thereof;
b) a continuous oil phase comprising one or more oil-soluble compounds each having a function selected from the group consisting of antioxidant, metal chelator, pH regulator, POV remediant, and combinations thereof;
c) a surfactant; and
d) a water-miscible solvent.

Embodiment 40. The water-in-oil microemulsion of embodiment 39, wherein the discontinuous water phase is 0.1 to 30 wt %, the continuous oil phase is 1 to 50 wt %, and the surfactant is 10 to 90 wt %, relative to the total weight of the microemulsion.

Embodiment 41. The water-in-oil microemulsion according to embodiment 39 or 40, wherein the surfactant is selected from the group consisting of sorbitan fatty acid ester, sorbitan monoester, sorbitan monolaurate, sorbitan monooleate, polyoxyethylene sorbitan fatty acid ester, polyoxyethylene sorbitan monoester, polyoxyethylene (20) sorbitan monolaurate, polyoxyethylene (20) sorbitan monooleate; polyglyceryl fatty acid esters, glyceryl monooleate, polyglyceryl-3 caprate, polyglyceryl-4 caprate, polyglyceryl-6 caprylate, polyglyceryl-3 cocoate, polyglyceryl-6 ricinoleate, polyglyceryl-10 laurate; a C₆-C₂₂ alkyl polyglucoside, a C₆-C₂₂ alkyl xyloside, fatty acids, lecithin, phosphatidylcholine, lysolecithin, natural and modified biosurfactants, such as saponins and glycolipids, for example, sophorolipids, mannosylerythritollipids and rhamnolipids; mixed propoxylated and ethoxylated aliphatic alcohols, linear and branched, primary and secondary alcohols with a chain length containing a total number of carbon atoms between 8 and 18 modified with a polyoxyethylene group carrying between 1 and 40 PEG units, PEG-modified hydrogenated castor oil carrying between 20 and 60 PEG units, triblock copolymers comprising blocks of polyethyleneglycol and polypropyleneglycol, and a combination thereof.

Embodiment 42. The water-in-oil microemulsion according to any one of embodiments 39 to 41, wherein the surfactant is selected from the group consisting of sorbitan monolaurate and polyoxyethylene (20) sorbitan monolaurate, and combinations thereof.

Embodiment 43. The water-in-oil microemulsion according to any one of embodiments 39 to 42, wherein the surfactant is sorbitan monolaurate.

Embodiment 44. The water-in-oil microemulsion according to any one of embodiments 39 to 43, wherein the continuous oil phase comprises an n-alkane, an iso-alkane, a cycloalkane, an n-alkene, an iso-alkene, a cycloalkene, an alkyl ester, a medium chain triglyceride, a silicon oil, a vegetable oil, a mineral oil, a paraffine, a perfume raw material, or a combination thereof.

Embodiment 45. The water-in-oil microemulsion according to any one of embodiments 39 to 44, wherein the discontinuous water phase comprises the pH regulator, wherein the pH regulator is water-soluble.

Embodiment 46. The water-in-oil microemulsion according to any one of embodiments 39 to 45, wherein the discontinuous water phase comprises the antioxidant and/or POV remediant, wherein the antioxidant and/or POV remediant are/is water-soluble.

Embodiment 47. The water-in-oil microemulsion according to any one of embodiments 39 to 46, wherein the discontinuous water phase comprises the metal chelator, wherein the metal chelator is water-soluble.

Embodiment 48. The water-in-oil microemulsion according to any one of embodiments 39 to 47, wherein the continuous oil phase comprises the antioxidant and/or POV remediant, wherein the antioxidant and/or POV remediant are/is oil-soluble.

Embodiment 49. The water-in-oil microemulsion according to any one of embodiments 39 to 48, wherein the water-miscible solvent is selected from the group consisting of C₁-C₆ alcohols, such as ethanol, n-propanol, 2-propanol, and 1-butanol; diols, such as 1,2-propanediol, 1,2-butanediol, 1,2-pentanediol, 1,2-hexanediol, 1,3-propanediol, 1,3-butanediol, 2,3-butanediol, and 2-methyl-2,4-pentanediol; polyalkylene glycols, such as dipropylene glycol; polyols, such as glycerol; triethyl citrate, isopropylidene glycerol, ethyl lactate, and combinations thereof.

Embodiment 50. The fragrance composition according to any one of embodiments 1 to 37, wherein the composition comprises less than or equal to about 0.9 wt %, less than or equal to about 0.8 wt %, less than or equal to about 0.7 wt %, less than or equal to about 0.6 wt %, less than or equal to about 0.5 wt %, less than or equal to about 0.4 wt %, less than or equal to about 0.3 wt %, less than or equal to about 0.2 wt % water, less than or equal to about 0.1 wt %, or less than or equal to about 0.05 wt % water, relative to the total weight of the composition.

Embodiment 51. A consumer product comprising the fragrance composition according to any one of embodiments 1 to 37 and 50, the solution according to embodiment 38, or the water-in-oil microemulsion according to any one of embodiments 39 to 49.

Embodiment 52. The consumer product according to embodiment 51, wherein the consumer product is a perfume, typically a fine fragrance, a fabric care product, a body-care product, a cosmetic preparation, a skin-care product, an air care product, or a home care product.

Embodiment 53. The consumer product according to embodiment 51 or 52, wherein the consumer product is an eau de toilette, a hydro-alcoholic solution, an ethanol free eau de toilette, a splash, eau de parfum, a cologne, a shave or after-shave lotion, a liquid or solid or unit-dose detergent, a fabric softener, a solid or liquid fabric scent-booster, a fabric refresher, an ironing water, a paper, a bleach, a carpet cleaners, curtain-care products, a shampoo, a leave-on or rinse-off hair conditioner, a coloring preparation, a color care product, a hair shaping product, a dental care product, a disinfectant, an intimate care product, a hair spray, a vanishing cream, a deodorant or antiperspirant, hair remover, tanning or sun product, a nail product, a skin cleansing, a makeup, a perfumed soap, a shower or bath mousse, oil or gel, or a foot/hand care products, a hygiene product, an air freshener, a “ready to use” powdered air freshener, a mold remover, a furniture care product, a wipe, a dish detergent or hard-surface detergent, a leather care product, or a car care product.

Embodiment 54. Use of the stabilizing mixture defined in any one of embodiments 1 to 37 and 50, the solution according to embodiment 38, or the water-in-oil microemulsion according to any one of embodiments 39 to 49, to enhance the oxidative stability of a fragrance oil in a fragrance composition or a consumer product.

Embodiment 55. The fragrance composition of embodiment 1 or 2, wherein the stabilizing mixture does not comprise citric acid.

Embodiment 56. The fragrance composition of embodiment 1 or 2, wherein the stabilizing mixture does not comprise tetramethylhydroxypiperidinol.

The compositions, products, and uses, according to the present disclosure are further illustrated by the following non-limiting examples.

### EXAMPLES

### Example 1. Compositions According to the Present Disclosure

Comparative compositions and inventive compositions according to some aspects of the present disclosure were prepared by combining the components shown in Table 1 in the amounts listed in Tables 2, 3, and 3a below. The composition of the fragrance oil used is provided in Table 4.

TABLE 1

| Component | Chemical description | Function |
|---|---|---|
| Covi-Ox ® T-90 EU C ¹⁾ | mixture of tocopherols | Antioxidant |
| Vitamin E | mixture of tocopherols | Antioxidant |
| Dermofeel ® TOCO 70 ²⁾ | mixture of tocopherols | Antioxidant |
| BHT | 3,5-di-tert-butyl-4- | Antioxidant |
| | hydroxytoluene | |
| Tinogard ® DA ³⁾ | didodecyl 3,3′- | Antioxidant |
| | thiodipropionate | |
| Tetronic ® 1301 ⁴⁾ | Poloxamine | Metal chelating |
| | | agent |
| Wayfos A | phosphoric acid, bis(2- | Metal chelating |
| | ethylhexyl) ester | agent |
| Dermofeel ® PA-12 ⁵⁾ | Phytic acid sodium salt | Metal chelating |
| | | agent |
| AKG | alpha-ketoglutaric acid | POV remediant |
| DEC | diethyl citrate | pH regulator |
| TEA | triethanolamine | pH regulator |
| NaHCO₃ | sodium bicarbonate | pH regulator |
| NaOH | sodium hydroxide | pH regulator |
| NaH₂PO₄ | monosodium phosphate | pH regulator |
| Na₂HPO₄ | disodium phosphate | pH regulator |
| Span ™ 20 ⁶⁾ | sorbitan monolaurate | Surfactant |
| ECO Tween ™ 20 ⁷⁾ | PEG-20 sorbitan | Surfactant |
| | monolaurate | |
| IPM | isopropyl myristate | Solvent |
| Cetiol ® Ultimate ⁸⁾ | undecane (and) | Solvent |
| | tridecane | |
| A-Leen ® 5 ⁹⁾ | 1,2-pentanediol | Solvent |
| Zemea ® Propanediol ¹⁰⁾ | 1,3-propanediol | Solvent |
| DIPG | dipropylene glycol | Solvent |
| DI water | deionized water | Solvent |

¹⁾ From BASF
²⁾ From Evonik
³⁾ From BASF
⁴⁾ From BASF
⁵⁾ From Evonik
⁶⁾ From Croda
⁷⁾ From Croda
⁸⁾ From BASF
⁹⁾ From Minasolve
¹⁰⁾ From DuPont Tate & Lyle

TABLE 2

| | Mixture | Mixture | Mixture | Mixture | Mixture | Mixture |
|---|---|---|---|---|---|---|
| Component | C1 | C2 | C3 | C4 | 5 | 6 |
| Covi-Ox ® | 100 | | | | | |
| T-90 EU C | | | | | | |
| Vitamin E | | 100 | | | | 6 |
| Dermofeel ® | | | 100 | 11.1 | | |
| TOCO 70 | | | | | | |
| BHT | | | | | 7 | |
| Tinogard ® | | | | | | 2.5 |
| DA | | | | | | |
| Tetronic ® | | | | | 10 | 30 |
| 1301 | | | | | | |
| Wayfos A | | | | | 0.08 | 0.04 |
| DEC | | | | | 2 | |
| TEA | | | | | 3 | |
| Span ™ 20 | | | | 22.2 | | |
| ECO | | | | 33.3 | | |
| Tween ™ 20 | | | | | | |
| IPM | | | | | | 61.46 |
| Cetiol ® | | | | 11.2 | | |
| Ultimate | | | | | | |
| A-Leen ® 5 | | | | 22.2 | | |
| DIPG | | | | | 72.92 | |
| DI water | | | | | 5 | |

TABLE 3

| | Mixture | Mixture | Mixture | Mixture | Mixture | Mixture |
|---|---|---|---|---|---|---|
| Component | 7 | 8 | 9 | 10 | 11 | 12 |
| Dermofeel ® | 6 | 10 | 10 | 10 | 6 | 6 |
| TOCO 70 | | | | | | |
| AKG | 0.25 | 0.25 | 0.25 | 0.25 | | 0.25 |
| NaHCO₃ | 0.4 | 0.4 | | | 0.4 | 0.4 |
| NaOH | 0.4 | 1.5 | | | | 1.5 |
| Span ™ 20 | 25 | 20 | 20 | 20 | 20 | 20 |
| ECO | 38 | 30 | 30 | 30 | 33 | 33 |
| Tween ™ 20 | | | | | | |
| Cetiol ® | 11 | 10 | 10 | 10 | 11 | 11 |
| Ultimate | | | | | | |
| A-Leen ® 5 | | 20 | 20 | 20 | 20 | 20 |
| Zemea ® | 10 | | | | | |
| Propanediol | | | | | | |
| DI water | 8.95 | 7.85 | 9.75 | 9.75 | 9.6 | 7.85 |
| pH (of | 7.94 | 8.52 | 1.53 | 11.76 | 8.61 | 8.52 |
| aqueous phase) | | | | | | |

TABLE 3a

| | Mixture | Mixture | Mixture | Mixture | Mixture |
|---|---|---|---|---|---|
| Component | 13 | 14 | 15 | 16 | 17 |
| Dermofeel ® TOCO 70 | 10 | 10 | 10 | 10 | 10 |
| Dermofeel ® PA-12 | | | | 0.01 | 0.01 |
| NaH₂PO₄ | 0.39 | 0.36 | 0.28 | 0.28 | 0.36 |
| Na₂HPO₄ | 0.02 | 0.06 | 0.15 | 0.15 | 0.06 |
| Span ™ 20 | 50 | 50 | 50 | 50 | 50 |
| Cetiol ® Ultimate | 10 | 10 | 10 | 10 | 5 |
| A-Leen ® 5 | 20 | 20 | 20 | 20 | 25 |
| DI water | 9.59 | 9.58 | 9.57 | 9.56 | 9.57 |

TABLE 4

| Ingredient | % w/w |
|---|---|
| Benzyl acetate | 0.60 |
| Geranyl acetate | 0.48 |
| Linalyl acetate | 2.77 |
| Anisaldehyde | 0.53 |
| Hexylcinnamic aldehyde | 3.06 |
| Ambrox ® ¹⁾ | 0.96 |
| Iso E Super | 2.41 |
| Ethylene brassylate | 31.40 |
| Beta dihydroionone | 1.56 |
| Citronellol | 0.55 |
| 4-Cyclohexyl-2-methyl-2-butanol | 3.49 |
| DIPG | 10.18 |
| Exaltolide ® ²⁾ | 2.89 |
| Tetrahydro-2-isobutyl-4-methyl- | 3.01 |
| 4(2H)-pyranol | |
| 4,6,6,7,8,8-hexamethyl-1,3,4,6,7,8- | 7.70 |
| hexahydrocyclopenta[g]isochromene | |
| Geraniol | 0.72 |
| Habanolide ® ³⁾ | 0.84 |
| Hedione ® ⁴⁾ | 15.16 |
| 3-(1,3-benzodioxol-5-yl)-2- | 0.84 |
| methylpropanal | |
| Helvetolide ® ⁵⁾ | 1.08 |
| Hydroxycitronellal | 1.44 |
| Methylionone | 0.72 |
| Linalol | 0.55 |
| Mandarine oil | 1.44 |
| Mayol ® ⁶⁾ | 1.56 |
| Patchouli oil | 0.60 |
| Phenethylol | 0.48 |
| Benzyl salicylate | 0.79 |
| 1-(3,5,5,6,8,8-hexamethyl-5,6,7,8- | 0.96 |
| tetrahydro-2-naphthalenyl)ethanone | |
| Methyl cedryl ketone | 1.23 |

¹⁾ (−)-(3aR,5aS,9aS,9bR)-3a,6,6,9a-tetramethyldodecahydronaphtho[2,1-b]furan
²⁾ Oxacyclohexadecan-2-one
³⁾ 1-Oxa-12-cyclohexadecen-2-one
⁴⁾ Methyl 2-((1RS,2RS)-3-oxo-2-pentylcyclopentyl)acetate
⁵⁾ (+)-2-{(1S)-1-[(1R)-3,3-dimethylcyclohexyl]ethoxy}-2-methylpropyl propionate
⁶⁾ 7-p-Menthanol

### Example 2. Evaluation of Fragrance Oil Stability

The stability of fragrance oil alone, fragrance oil combined with comparative mixtures C1-C4, and inventive fragrance compositions comprising stabilizing mixtures 5-7 according to Example 1 were evaluated by a method based on ASTM D7545 and described in MARTEAU C. et al., Oxidative degradation of fragrant aldehydes. Autoxidation by molecular oxygen, Tetrahedron 69 (2013) 2268-2275. The measuring principle is based on ASTM D7545 norm, which is a normalized method to study the oxidation stability of diesel, biodiesel and fuel mixtures. The induction period to determine the oxidation stability is measured and the closed system guarantees a high precision of the test results in a fraction of time compared to other oxidation and storage stability test methods.

Tests were conducted on a RapidOxy model 100 instrument (available from Anton Paar (Graz, Austria)). The instrument artificially accelerates the oxidation process by using increased temperature and oxygen pressure. Each sample was placed in contact with oxygen under defined pressure and temperature (typically 300 kPa at 140° C.). The induction time of the product was measured when the oxygen pressure drop reached a Δpₘₐₓ of 5%.

The induction times (in minutes) were used to calculate a value Aₒᵢₗ, which is the stabilizing efficiency of the stabilizing mixture on the fragrance oil, according to the equation:

$${A}_{oil} ( \% ) = \frac{{t}_{stab .} - {t}_{non - stab .}}{{t}_{non - stab .}} \times 100$$

wherein
t_{stab} is the Rapidoxy induction time of fragrance oil comprising the stabilizing mixture,
t_{non-stab} is the Rapidoxy induction time of fragrance oil without the stabilizing mixture.

The stability results of fragrance oil alone and fragrance oil combined with comparative mixtures C1-C4 are shown in Table 5a below. The stability results of inventive fragrance compositions comprising stabilizing mixtures 5-7 are shown in Table 5b below.

TABLE 5a

| | Comparative | Comparative | Comparative | Comparative | Comparative |
|---|---|---|---|---|---|
| | fragrance | fragrance | fragrance | fragrance | fragrance |
| | composition | composition | composition | composition | composition |
| | 1 | 2 | 3 | 4 | 5 |
| Stabilizing | — | mixture C1 | mixture C2 | mixture C3 | mixture C4 |
| mixture | | | | | |
| Stabilizing | — | 0.2 | 0.2 | 0.2 | 2 |
| mixture | | | | | |
| quantity | | | | | |
| (%) | | | | | |
| Fragrance | 100.0 | 99.8 | 99.8 | 99.8 | 98 |
| (%) | | | | | |
| Induction | 33.3 | 87.9 | 67.7 | 87.1 | 58.7 |
| time (min) | | | | | |
| Aₒᵢₗ (%) | | 163.8 | 103.2 | 161.5 | 76.2 |

TABLE 5b

| | Fragrance | Fragrance | Fragrance |
|---|---|---|---|
| | Composition | Composition | Composition |
| | 6 | 7 | 8 |
| Stabilizing | mixture 5 | mixture 6 | mixture 7 |
| mixture | | | |
| Stabilizing | 1 | 2 | 2 |
| mixture | | | |
| quantity | | | |
| (%) | | | |
| Fragrance | 99 | 98 | 98 |
| (%) | | | |
| Induction | 66.4 | 64.4 | 47.1 |
| time (min) | | | |
| Aₒᵢₗ (%) | 99.4 | 93.3 | 41.3 |

As shown in Tables 5a and 5b, all samples show longer induction times than the non-stabilized oil (Aₒᵢₗ>0), indicating that the presence of stabilizers, such as vitamin E, enhances the stability of fragrance oils.

### Example 3. Eau De Toilette (EdT) Stability

The overall oxidation of consumer products in the form of eau de toilette formulations was assessed by measuring the concentration of diethoxyethane (DEE) according to a published method (BLAKEWAY J. M. et al., Chemical reactions in perfume ageing, International Journal of Cosmetic Science 9, 203-214 (1987)). The presence of DEE is a general marker of oxidation in ethanol-containing formulations, such as EdTs. As EdTs age, oxidation of ethanol results in the formation of acetaldehyde, which reacts with ethanol to form the corresponding acetal, or diethoxyethane.

Gas chromatographic method coupled with mass spectrometry detection (GC/MS) was used (Agilent Technologies 7890A-5975C). Alcoholic solutions were directly injected into the chromatograph, then separated on the stationary phase of the column and finally detected by mass spectrometry as they elute from the column.

The DEE concentrations (in ppm) obtained by GC/MS were used to calculate a value A_{EdT}, which is the stabilizing efficiency of the stabilizing mixture in the EdT, according to the equation:
wherein:

$${A}_{EdT} ( \% ) = \frac{{c}_{non - stab .}^{DEE} - {c}_{stab .}^{DEE}}{{c}_{non - stab .}^{DEE}} \times 100$$

$${c}_{stab}^{DEE}$$

is the concentration of DEE in an EdT formulation comprising a stabilizing mixture, and

$${c}_{non - stab}^{DEE}$$

is the concentration of DEE in an EdT formulation without the stabilizing mixture.

The stability results are summarized in Tables 6 and 7 below.

TABLE 6

| | Comparative | Comparative | Comparative | Comparative |
|---|---|---|---|---|
| | EdT 9 | EdT 10 | EdT 11 | EdT 12 |
| Comparative fragrance | 10 | — | — | — |
| composition 1 (%) | | | | |
| Comparative fragrance | — | 10 | — | — |
| composition 2 (%) | | | | |
| Comparative fragrance | — | — | 10 | — |
| composition 3 (%) | | | | |
| Comparative fragrance | — | — | — | 10 |
| composition 4 (%) | | | | |
| Ethanol (%) | 81 | 81 | 81 | 81 |
| DI water (%) | 9 | 9 | 9 | 9 |
| DEE (ppm) in EdT after | 71.0 | 68.0 | 109.0 | 58.0 |
| 2 months at 50 °C | | | | |
| DEE (ppm) in EdT after | 68.0 | 48.0 | 76.0 | 47.0 |
| 2 months at 50 °C, | | | | |
| half-full | | | | |
| DEE (ppm) in EdT after | 229.0 | 136.0 | 184.0 | 156.0 |
| 2 months at 50 °C, | | | | |
| half-full, plus spring | | | | |
| A_{EdT} (%) after 2 months | — | 4.2 | −53.5 | 18.3 |
| at 50 °C | | | | |
| A_{EdT} (%) after 2 months | — | 29.4 | −11.8 | 30.9 |
| at 50 °C, half-full | | | | |
| A_{EdT} (%) after 2 months | — | 40.6 | 19.7 | 31.9 |
| at 50 °C, half-full, | | | | |
| plus spring | | | | |

TABLE 7

| | Comparative | Inventive | Inventive | Inventive |
|---|---|---|---|---|
| | EdT 13 | EdT 14 | EdT 15 | EdT 16 |
| Comparative fragrance | 10 | — | — | — |
| composition 5 | | | | |
| Fragrance composition 6 | — | 10 | — | — |
| Fragrance composition 7 | — | — | 10 | — |
| Fragrance composition 8 | — | — | — | 10 |
| Ethanol (%) | 81 | 81 | 81 | 81 |
| DI water (%) | 9 | 9 | 9 | 9 |
| DEE (ppm) in EdT after 2 | 59.0 | 14.0 | 24.0 | 32.0 |
| months at 50 °C | | | | |
| DEE (ppm) in EdT after 2 | 40.0 | 15.0 | 21.0 | 27.0 |
| months at 50 °C, half-full | | | | |
| DEE (ppm) in EdT after 2 | 95.0 | 12.0 | 28.0 | 52.0 |
| months at 50 °C, half-full, | | | | |
| plus spring | | | | |
| A_{EdT} (%) after 2 months at | 16.9 | 80.3 | 66.2 | 54.9 |
| 50 °C | | | | |
| A_{EdT} (%) after 2 months at | 41.2 | 77.9 | 69.1 | 60.3 |
| 50 °C, half-full | | | | |
| A_{EdT} (%) after 2 months at | 58.5 | 94.8 | 87.8 | 77.3 |
| 50 °C, half-full, plus spring | | | | |

Comparative EdT's containing mixtures C1-C4, which only contain vitamin E, show limited stabilizing efficiency A_{EdT} or worse stability compared to the non-stabilized oil (negative values).

Inventive EdT's having fragrance compositions comprising stabilizing mixtures 5-7, which contain antioxidants, pH regulators, plus metal chelating agents or POV remediants, showed high stabilizing efficiency compared to the non-stabilized fragrance oil, and significantly higher values than comparative samples containing mixtures C1-C4.

### Example 4. Fragrance Oil Stability and pH

Compositions according to some aspects of the present disclosure having varying pH were evaluated according to the method described in Example 2. The results are summarized in Table 8 below.

TABLE 8

| | Fragrance | Fragrance | Fragrance |
|---|---|---|---|
| | composition | composition | composition |
| | 17 | 18 | 19 |
| Stabilizing mixture | Mixture 8 | Mixture 9 | Mixture 10 |
| Stabilizing mixture | 2 | 2 | 2 |
| quantity (%) | | | |
| Fragrance (%) | 98 | 98 | 98 |
| pH (of aqueous phase) | 8.5 | 1.5 | 11.8 |
| Induction time | 45.6 | 61.2 | 48.9 |
| Rapidoxy (min) | | | |
| Aₒᵢₗ (%) | 37.0 | 83.6 | 46.8 |

Fragrance compositions 17-19 all show a stabilizing effect on the fragrance oil compared to the non-stabilized oil (Comparative fragrance composition 1).

### Example 5. EdT Stability and pH

Consumer products in the form of EdT formulations according to some aspects of the present disclosure having varying pH were evaluated according to the method described in Example 3. The results are summarized in Table 9 below.

TABLE 9

| | Inventive | Inventive | Inventive |
|---|---|---|---|
| | EdT 20 | EdT 21 | EdT 22 |
| Fragrance composition 17 (%) | 10 | — | — |
| Fragrance composition 18 (%) | — | 10 | — |
| Fragrance composition 19 (%) | — | — | 10 |
| Ethanol (%) | 81 | 81 | 81 |
| DI water (%) | 9 | 9 | 9 |
| pH (of aqueous phase) | 8.5 | 1.5 | 11.8 |
| DEE (ppm) in EdT after 2 | 30.0 | 48.0 | 44.0 |
| months at 50 °C | | | |
| DEE (ppm) in EdT after 2 | 27.0 | 42.0 | 28.0 |
| months at 50 °C, half-full | | | |
| DEE (ppm) in EdT after 2 | 63.0 | 155.0 | 88.0 |
| months at 50 °C, half-full, plus | | | |
| spring | | | |
| A_{EdT} (%) after 2 months at 50 °C | 57.7 | 32.4 | 38.0 |
| A_{EdT} (%) after 2 months at 50 °C, | 60.3 | 38.2 | 58.8 |
| half-full | | | |
| A_{EdT} (%) after 2 months at 50 °C, | 72.5 | 32.3 | 61.6 |
| half-full, plus spring | | | |

Inventive EdT's 20-22 all show a stabilizing effect of the EdT compared to the non-stabilized EdT (Comparative EdT 9). The highest stabilizing level was achieved at pH 8.5.

### Example 6. Effect of POV Remediant on Oil Stability

Compositions according to some aspects of the present disclosure were prepared with a pH regulator in the aqueous phase and in the absence or presence of a POV remediant, AKG, and were evaluated according to the method described in Example 2. The results are summarized in Table 10 below.

TABLE 10

| | Fragrance | Fragrance |
|---|---|---|
| | composition | composition |
| | 23 | 24 |
| Stabilizing mixture | Mixture 11 | Mixture 12 |
| Stabilizing mixture quantity (%) | 2 | 2 |
| Fragrance (%) | 98 | 98 |
| Induction time Rapidoxy (min) | 46.0 | 45.3 |
| Aₒᵢₗ (%) | 38.2 | 35.9 |

Fragrance compositions 23 and 24 both show similar stabilizing effect of the oil compared to the non-stabilized oil (Comparative fragrance composition 1).

### Example 7. Effect of POV Remediant on EdT Stability

Consumer products in the form of EdT formulations according to some aspects of the present disclosure were prepared with a pH regulator in the aqueous phase and in the absence or presence of a POV remediant, AKG, and were evaluated according to the method described in Example 3. The results are summarized in Table 11 below.

TABLE 11

| | Inventive | Inventive |
|---|---|---|
| | EdT 25 | EdT 26 |
| Fragrance composition 23 (%) | 10 | — |
| Fragrance composition 24 (%) | — | 10 |
| Ethanol (%) | 81 | 81 |
| DI water (%) | 9 | 9 |
| DEE (ppm) in EdT after 2 months | 37.0 | 30.0 |
| at 50 °C | | |
| DEE (ppm) in EdT after 2 months | 28.0 | 22.0 |
| at 50 °C, half-full | | |
| DEE (ppm) in EdT after 2 months at | 57.0 | 58.0 |
| 50 °C, half-full, plus spring | | |
| A_{EdT} (%) after 2 months at 50 °C | 47.9 | 57.7 |
| A_{EdT} (%) after 2 months at 50 °C, | 58.8 | 67.6 |
| half-full | | |
| A_{EdT} (%) after 2 months at 50 °C, | 75.1 | 74.7 |
| half-full, plus spring | | |

Inventive EdT's 25 and 26 both show a stabilizing effect in the EdTs containing them compared to the non-stabilized EdT (Comparative EdT 9), but the effect is systematically higher in the presence of AKG.

### Example 8. Inventive Compositions on Oil Stability

Compositions according to some aspects of the present disclosure were prepared with phosphate salts as pH regulators in the aqueous phase and in the absence or presence of a metal chelating agent, phytic acid sodium salt, and were evaluated according to the method described in Example 2. The results are summarized in Table 12 below.

TABLE 12

| | Fragrance | Fragrance | Fragrance | Fragrance | Fragrance |
|---|---|---|---|---|---|
| | composition | composition | composition | composition | composition |
| | 27 | 28 | 29 | 30 | 31 |
| Stabilizing | mixture 13 | mixture 14 | mixture 15 | mixture 16 | mixture 17 |
| mixture | | | | | |
| Stabilizing | 2.0 | 2.0 | 2.0 | 2.0 | 2.0 |
| mixture | | | | | |
| quantity (%) | | | | | |
| Fragrance | 98.0 | 98.0 | 98.0 | 98.0 | 98.0 |
| (%) | | | | | |
| Induction | 48.41 | 47.60 | 50.16 | 51.81 | 58.33 |
| time | | | | | |
| Rapidoxy | | | | | |
| (min) | | | | | |
| Aₒᵢₗ | 45.38 | 42.94 | 50.63 | 55.59 | 75.17 |

### Example 9. Inventive Compositions on EdT Stability

Consumer products in the form of EdT formulations according to some aspects of the present disclosure were prepared with phosphate salts as pH regulators in the aqueous phase and in the absence or presence of a metal chelating agent, phytic acid sodium salt, and were evaluated according to the method described in Example 3. The results are summarized in Table 13 below.

TABLE 13

| | Inventive | Inventive | Inventive | Inventive | Inventive |
|---|---|---|---|---|---|
| | EdT 32 | EdT 33 | EdT 34 | EdT 35 | EdT 36 |
| Fragrance | 10 | — | — | — | — |
| composition 27 (%) | | | | | |
| Fragrance | — | 10 | — | — | — |
| composition 28 (%) | | | | | |
| Fragrance | — | — | 10 | — | — |
| composition 29 (%) | | | | | |
| Fragrance | — | — | — | 10 | — |
| composition 30 (%) | | | | | |
| Fragrance | — | — | — | — | 10 |
| composition 31 (%) | | | | | |
| Ethanol (%) | 81 | 81 | 81 | 81 | 81 |
| DI water (%) | 9 | 9 | 9 | 9 | 9 |
| DEE (ppm) in EdT | 50 | 65 | 30 | 60 | 65 |
| after 2 months at | | | | | |
| 50 °C | | | | | |
| DEE (ppm) in EdT | 45 | 45 | 45 | 55 | 60 |
| after 2 months at | | | | | |
| 50 °C, half-full | | | | | |
| DEE (ppm) in EdT | 40 | 30 | 40 | 55 | 40 |
| after 2 months at | | | | | |
| 50 °C, half-full, | | | | | |
| plus spring | | | | | |
| A_{EDT} (%) after 2 | 30 | 8 | 58 | 15 | 8 |
| months at 50 °C | | | | | |
| A_{EDT} (%) in after 2 | 34 | 34 | 34 | 19 | 12 |
| months at 50 °C, | | | | | |
| half-full | | | | | |
| A_{EDT} (%) after 2 | 83 | 87 | 83 | 76 | 83 |
| months at 50 °C, | | | | | |
| half-full, plus spring | | | | | |

The disclosed subject matter has been described with reference to specific details of particular embodiments thereof. It is not intended that such details be regarded as limitations upon the scope of the disclosed subject matter except insofar as and to the extent that they are included in the accompanying claims.

Therefore, the exemplary embodiments described herein are well adapted to attain the ends and advantages mentioned as well as those that are inherent therein. The particular embodiments disclosed above are illustrative only, as the exemplary embodiments described herein may be modified and practiced in different but equivalent manners apparent to those of ordinary skill in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the exemplary embodiments described herein. The exemplary embodiments described herein illustratively disclosed herein suitably may be practiced in the absence of any element that is not specifically disclosed herein and/or any optional element disclosed herein.

1. A fragrance composition comprising:
a) a fragrance oil, and
b) a stabilizing mixture comprising at least 2 ingredients, each having a function selected from the group consisting of antioxidant, metal chelator, pH regulator, and POV remediant,
wherein the at least 2 ingredients, do not have the same function, and
wherein the composition contains less than about 1 wt % water, relative to the total weight of the composition.

2. The fragrance composition according to claim 1, wherein the antioxidant is selected from the group consisting of ascorbic acid, gallic acid, ferulic acid, caffeic acid, tannic acid, chlorogenic acid, ellagic acid, rosmarinic acid, a polyphenol, tetramethylhydroxypiperidinol, vitamin E, α-tocopherol, β-tocopherol, γ-tocopherol, δ -tocopherol, ascorbyl palmitate, ascorbyl glucoside, natural extracts, typically rosemary extract, curcumin, tetrahydrocurcumin, ethyl ferulate, ethyl gallate, resveratrol, 3,5-di-tert-butyl-4-hydroxytoluene, pentaerythrityl tetra-di-t-butyl hydroxyhydrocinnamate, 2,2′-methylenebis(6-tert-butyl-4-methyl-phenol), didodecyl 3,3′-thiodipropionate, tetramethylhydroxypiperidinol, ubiquinone, ubiquinol, and a combination thereof.

3. The fragrance composition according to claim 1, wherein the metal chelator is selected from the group consisting of phytic acid, or a salt thereof, citric acid, tetrasodium iminodisuccinate, tetrasodium glutamate diacetate, trisodium dicarboxymethyl alaninate, ethylenediaminetetraacetic acid, trans-1,2-diaminocyclohexane-N,N,N′,N′-tetraacetic acid, a poloxamine, phosphoric acid bis(2-ethylhexyl) ester, and a combination thereof.

4. The fragrance composition according to claim 1, wherein the pH regulator is selected from the group consisting of sodium, potassium, and ammonium salts of carbonate, bicarbonate, phosphate, monohydrogenphosphate, dihydrogenphosphate, lactate, citrate, succinate, and hydroxide; dimethylglucamine, methylglucamine, tetrahydroxypropyl ethylenediamine, glycine, mono- and diethyl citrate, ethyl succinate, triethanolamine, an alkyldiethanoleamine, tricine, tris buffer, and a combination thereof.

5. The fragrance composition according to claim 1, wherein the POV remediant comprises an α-oxocarboxylic acid, or a salt thereof, or a 2-hydroxyketone.

6. The fragrance composition according to claim 1, wherein the stabilizing mixture is in the form of a solution or an emulsion.

7. The fragrance composition according to claim 6, wherein the stabilizing mixture is in the form of a water-in-oil microemulsion comprising:
a) a discontinuous aqueous phase comprising water;
b) a continuous oil phase;
c) a surfactant; and
d) a water-miscible solvent.

8. The fragrance composition according to claim 7, wherein the discontinuous water phase is 0.1 to 30 wt %, the continuous oil phase is 1 to 50 wt %, and the surfactant is 10 to 90 wt %, relative to the total weight of the microemulsion.

9. The fragrance composition according to claim 7, wherein the surfactant is selected from the group consisting of sorbitan fatty acid ester, sorbitan monoester, sorbitan monolaurate, sorbitan monooleate, polyoxyethylene sorbitan fatty acid ester, polyoxyethylene sorbitan monoester, polyoxyethylene (20) sorbitan monolaurate, polyoxyethylene (20) sorbitan monooleate; polyglyceryl fatty acid esters, glyceryl monooleate, polyglyceryl-3 caprate, polyglyceryl-4 caprate, polyglyceryl-6 caprylate, polyglyceryl-3 cocoate, polyglyceryl-6 ricinoleate, polyglyceryl-10 laurate; a C₆-C₂₂ alkyl polyglucoside, a C₆-C₂₂ alkyl xyloside, fatty acids, lecithin, phosphatidylcholine, lysolecithin, natural and modified biosurfactants, mixed propoxylated and ethoxylated aliphatic alcohols, linear and branched, primary and secondary alcohols with a chain length containing a total number of carbon atoms between 8 and 18 modified with a polyoxyethylene group carrying between 1 and 40 PEG units, PEG-modified hydrogenated castor oil carrying between 20 and 60 PEG units, triblock copolymers comprising blocks of polyethyleneglycol and polypropyleneglycol, and a combination thereof.

10. The fragrance composition according to claim 7, wherein the continuous oil phase comprises an n-alkane, an iso-alkane, a cycloalkane, an n-alkene, an iso-alkene, a cycloalkene, an alkyl ester, a medium chain triglyceride, a silicon oil, a vegetable oil, a mineral oil, a paraffine, a perfume raw material, or a combination thereof.

11. The fragrance composition according to claim 1, wherein the discontinuous water phase comprises the pH regulator, the metal chelator, the antioxidant, and/or the POV remediant wherein the pH regulator, the metal chelator, the antioxidant, and/or the POV remediant is/are water-soluble.

12. The fragrance composition according to claim 7, wherein the continuous oil phase comprises the antioxidant and/or POV remediant, wherein the antioxidant and/or POV remediant are/is oil-soluble.

13. The fragrance composition according to claim 7, wherein the water-miscible solvent is selected from the group consisting of C₁-C₆ alcohols, diols, polyalkylene glycols, polyols, triethyl citrate, isopropylidene glycerol, ethyl lactate, and combinations thereof.

14. A solution comprising:
one or more solvents, and
at least 2 ingredients, each having a function selected from the group consisting of antioxidant, metal chelator, pH regulator, and POV remediant,
wherein the at least 2 ingredients, do not have the same function.

15. A water-in-oil microemulsion comprising:
a) a discontinuous aqueous phase comprising water and one or more water-soluble compounds each having a function selected from the group consisting of antioxidant, metal chelator, pH regulator, POV remediant, and combinations thereof;
b) a continuous oil phase comprising one or more oil-soluble compounds each having a function selected from the group consisting of antioxidant, metal chelator, pH regulator, POV remediant, and combinations thereof;
c) a surfactant; and
d) a water-miscible solvent.

16. A consumer product, comprising the fragrance composition according to claim 1.

17. A method to enhance the oxidative stability of a fragrance oil in a fragrance composition or a consumer product, the method comprising using the stabilizing mixture defined in claim 1.
